SEQUENCE LISTING (12 sequences)

SEQ ID NO: 1 - protein, 118 aa, synthetic construct
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPS

SEQ ID NO: 2 - protein, 119 aa, synthetic construct
EEELQIIQPDKSVLVAAGETATLRCTITSLFPVGPIQWFRGAGPGRVLIYNQRQGPFPRVTTVSDTTKRNNMDFSIRIGNITPADAGTYYCIKFRKGSPDDVEFKSGAGTELSVRAKPS

SEQ ID NO: 3 - protein, 115 aa, synthetic construct
XXELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVR

SEQ ID NO: 4 - protein, 114 aa, synthetic construct
XXELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVR

SEQ ID NO: 5 - protein, 117 aa, synthetic construct
EEXLQVIQPDKXVXVAAGEXAXLXCTXTSLIPVGPIQWFRGAGPXRELIYNQKEGHFPRVTTVSXXDLTKRXNMDFXIXIXNITPADAGTYYCVKFRKGSPDDXEFKSGAGTELSVR

SEQ ID NO: 6 - protein, 215 aa, synthetic construct
ELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 224 aa, synthetic construct
PPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 8 - protein, 224 aa, synthetic construct
PPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 9 - protein, 224 aa, synthetic construct
PPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 346 aa, synthetic construct
EEELQIIQPDKSVLVAAGETATLRCTITSLFPVGPIQWFRGAGPGRVLIYNQRQGPFPRVTTVSDTTKRNNMDFSIRIGNITPADAGTYYCIKFRKGSPDDVEFKSGAGTELSVRAKPSAAAPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 8 aa, synthetic construct
GGGSGGGS

SEQ ID NO: 12 - protein, 13 aa, synthetic construct
ASTKGPSVFPLAP

(19) United States
(12) Patent Application Publication
(10) Pub. No.: US 2023/0365648 A1
(43) Pub. Date: Nov. 16, 2023

(54) SIRP-ALPHA FUSION POLYPEPTIDES WITH MODIFIED FC DOMAINS

(71) Applicant: Bitterroot Bio, Inc., Palo Alto, CA (US)

(72) Inventor: Jens-Peter VOLKMER, Recklinghausen (DE)

(21) Appl. No.: 18/189,152

(22) Filed: Mar. 23, 2023

Related U.S. Application Data
(60) Provisional application No. 63/323,417, filed on Mar. 24, 2022.

(51) Int. Cl. C07K 14/705; A61P 9/00

(52) U.S. Cl. CPC C07K 14/70503; A61P 9/00; C07K 2319/31; C07K 2319/74

(57) ABSTRACT

Provided herein are signal-regulatory protein α (SIRPα) fusion polypeptides comprising modified Fc domains, and methods of making and use thereof. The inclusion of the modified Fc domains results in pharmacokinetic and/or phamacodynamic improvements to the SIRPα fusion polypeptides. The compositions and methods described herein may be used to treat a variety of diseases, such as cardiovascular disease.

### CROSS REFERENCE TO RELATED APPLICATIONS

This application claims priority to U.S. Provisional Patent Application No. 63/323,417 filed on Mar. 24, 2022, the contents of which are incorporated by reference in their entirety.

### INCORPORATION BY REFERENCE OF SEQUENCE LISTING

The contents of the electronic sequence listing (BTRT_010_01WO_SeqList_ST26.xml; Size: 13,201 bytes; and Date of Creation: Mar. 21, 2023) are herein incorporated by reference in their entirety.

### BACKGROUND

Optimization of therapies comprising IgG Fc domains remains a challenge as these polypeptides can exhibit variable half-lives in the bloodstream. One example is signal-regulatory protein α (SIRPα) fusion polypeptides with IgG Fc domains, designed to engage the CD47 protein, which is applicable to a wide range of diseases benefiting from optimal pharmacological inhibition of CD47. Therefore, a need exists for signal-regulatory protein α (SIRPα)-IgG Fc therapies with improved pharmacokinetics and/or pharmacodynamics; provided herein are compositions and methods that address this need.

### SUMMARY

The present disclosure provides signal-regulatory protein α (SIRPα) fusion polypeptide treatments comprising modified Fc domains for improved pharmacokinetics and/or pharmacodynamics of the fusion polypeptide.

In some embodiments of the disclosure, provided is a fusion polypeptide comprising a signal-regulatory protein α (SIRPα) domain, and a modified Fc domain, wherein the modified Fc domain comprises one or more amino acid modifications relative to a wild type Fc domain, and wherein the inclusion of the modified Fc domain increases binding affinity to FcRn.

In some embodiments of the disclosure, inclusion of the modified Fc domain increases the half-life of the fusion polypeptide. In some embodiments of the disclosure, inclusion of the modified Fc domain increases the blood clearance time of the fusion polypeptide.

In some embodiments of the disclosure, the inclusion of the modified Fc domain increases the binding affinity of the fusion polypeptide to a CD47 protein. In some embodiments of the disclosure, the inclusion of the modified Fc domain lowers the EC₅₀ of the fusion polypeptide.

In some embodiments, inclusion of the modified Fc domain lowers the effective dose of the fusion polypeptide necessary to achieve a therapeutic effect in a subject. In some embodiments, inclusion of the modified Fc domain lowers the dosage frequency of the fusion polypeptide in a subject.

In some embodiments, inclusion of the modified Fc domain decreases the toxicity of the fusion polypeptide. In some embodiments, inclusion of the modified Fc domain decreases the antibody dependent cellular cytotoxicity (ADCC).

In some embodiments, the inclusion of the modified Fc domain increases effector function of the fusion polypeptide. In some embodiments, inclusion of the modified Fc domain increases phagocytosis by a macrophage. In some embodiments, inclusion of the modified Fc domain increases the interaction of the fusion polypeptide with a cell expressing a CD47 protein. In some embodiments, inclusion of the modified Fc domain increases endocytosis of a CD47 protein.

In some embodiments, inclusion of the modified Fc domain increases degradation of a CD47 protein. In some embodiments, the CD47 protein is a human or mouse CD47 protein. In some embodiments, inclusion of the modified Fc domain reduces aggregation of the fusion polypeptide. In some embodiments, inclusion of the modified Fc domain improves purification of the polypeptide.

In some embodiments, the modified Fc domain comprises the IgG4 Fc domain amino acid sequence of SEQ ID NOS: 7 or 8 or an amino acid sequence with at least 70%, at least 75%, at least 80%, at least 85%, at least 90%, at least 95%, at least 96%, at least 97%, at least 98%, or at least 99% sequence identity thereto.

In some embodiments, the modified Fc domain comprises one or more substitutions selected from the group consisting of T250Q, M252Y, S254T, T256E, S267E, N325S, L328F, N343S, M428L, N434F, and H443K relative to SEQ ID NOS: 7 or 8, according to the EU numbering scheme. In some embodiments, the modified Fc domain comprises one or more substitutions selected from the group consisting of T250Q-M428L, M252Y-S254T-T256E, M428L-N434S, S267E-L328F, N325S-L328F, and H433K-N434F, relative to SEQ ID NOS: 7 or 8 according to the EU numbering scheme. In some embodiments, the modified Fc domain comprises SEQ ID NO: 9 or an amino acid sequence with at least 70%, at least 75%, at least 80%, at least 85%, at least 90%, at least 95%, at least 96%, at least 97%, at least 98%, or at least 99% sequence identity thereto.

In some embodiments, the modified Fc domain comprises the IgG1 Fc domain amino acid sequence of SEQ ID NO: 6 or an amino acid sequence with at least 70%, at least 75%, at least 80%, at least 85%, at least 90%, at least 95%, at least 96%, at least 97%, at least 98%, or at least 99% sequence identity thereto. In some embodiments, the modified Fc domain comprises one or more substitutions selected from the group consisting of V215A, G236A, S239D, and I332E relative to SEQ ID NO: 6, according to the EU numbering scheme. In some embodiments, the modified Fc domain comprises one or more substitutions selected from the group consisting of T250Q, M252Y, S254T, T256E, S267E, N325S, L328F, N343S, M428L, H433K, and N434F relative to SEQ ID NO: 6, according to the EU numbering scheme. In some embodiments, the modified Fc domain comprises one or more substitutions selected from the group consisting of T250Q-M428L, M252Y-S254T-T256E, M428L-N434S, S267E-L328F, N325S-L328F, and H433K-N434F relative to SEQ ID NO: 6, according to the EU numbering scheme.

In some embodiments, the modified Fc domain is a modified human IgG1, IgG2, IgG3, or IgG4 domain.

In some embodiments, the SIRPα domain comprises the amino acid sequence of SEQ ID NO: 1 or an amino acid sequence with at least 70%, at least 75%, at least 80%, at least 85%, at least 90%, at least 95%, at least 96%, at least 97%, at least 98%, or at least 99% sequence identity thereto. In some embodiments, the SIRPα domain comprises one or more of the following mutations relative to SEQ ID NO: 1: V6I, S14L, S20T, I22T, H24R, V27I, I31F, A45G, E47V, K53R, E54Q, H56P, S66T, E70N, S77R, V92I, and/or a duplication of the D100 residue. In some embodiments, the SIRPα domain comprises the amino acid sequence of SEQ ID NO: 2 or an amino acid sequence with at least 70%, at least 75%, at least 80%, at least 85%, at least 90%, at least 95%, at least 96%, at least 97%, at least 98%, or at least 99% sequence identity thereto.

In some embodiments, the modified Fc domain comprises any of SEQ ID NOS: 6-9, or an amino acid sequence with at least 70%, at least 75%, at least 80%, at least 85%, at least 90%, at least 95%, at least 96%, at least 97%, at least 98%, or at least 99% sequence identity thereto; and the SIRPα domain comprises SEQ ID NO: 1 or SEQ ID NO: 2 or an amino acid sequence with at least 70%, at least 75%, at least 80%, at least 85%, at least 90%, at least 95%, at least 96%, at least 97%, at least 98%, or at least 99% sequence identity thereto. In some embodiments, the modified Fc domain comprises SEQ ID NO: 9 or an amino acid sequence with at least 70%, at least 75%, at least 80%, at least 85%, at least 90%, at least 95%, at least 96%, at least 97%, at least 98%, or at least 99% sequence identity thereto; and the SIRPα domain comprises SEQ ID NO: 2 or an amino acid sequence with at least 70%, at least 75%, at least 80%, at least 85%, at least 90%, at least 95%, at least 96%, at least 97%, at least 98%, or at least 99% sequence identity thereto.

In some embodiments, provided herein is a method of treating a disease in a subject in need thereof, comprising administering a SIRPα fusion polypeptide to the subject. In some embodiments, the disease is a cardiovascular disease. In some embodiments, the fusion polypeptide is administered subcutaneously.

In some embodiments, provided herein is a method of increasing phagocytosis by macrophages comprising contacting a population of macrophages with a SIRPα fusion polypeptide. In some embodiments, the macrophage is a human macrophage.

In some embodiments, provided herein is a nucleotide encoding a SIRPα fusion polypeptide.

### BRIEF DESCRIPTION OF THE DRAWINGS

FIG. 1 is a chart showing binding affinity to different CD47 proteins for an example SIRPα fusion polypeptide with a YTE Fc domain mutation (Construct 67), relative to a SIRPα fusion polypeptide with a wild type Fc domain and a MIAP410 anti-CD47 mononoclonal antibody.

FIGS. 2A and 2B show example individual CD47 binding assay runs for SIRPα fusion polypeptides Construct 50 (FIG. 2A) and Construct 67 (FIG. 2B).

FIG. 3 shows the EC₅₀ effect for SIRPα fusion polypeptides Construct 50 and Construct 67 for the amount of construct bound to human CD45 negative red blood cells in vitro.

FIGS. 4A and 4B show the level of phagocytosis induced by SIRPα fusion polypeptides Construct 67, and Construct 50, and an anti-CD47 antibody. FIG. 4A and FIG. 4B each show different human donor results.

FIGS. 5A and 5B show the effect on antibody dependent cellular cytotoxicity (ADCC) of SIRPα fusion polypeptides Construct 67 and Construct 50 relative to an antibody to CD-20. FIG. 5A shows that Construct 67 and Construct 50 demonstrated minimal ADCC compared to the anti-CD20 antibody. FIG. 5B is an inset of FIG. 5A, rescaled to show the difference between Construct 67 containing the YTE mutation in the Fc domain and Construct 50, containing the wild type Fc domain.

### DETAILED DESCRIPTION

Provided herein are signal-regulatory protein α (SIRPα) fusion polypeptide treatments comprising modified Fc domains, wherein the inclusion of the modified Fc domain improves pharmacokinetics and/or pharmacodynamics of the fusion polypeptide. The signal-regulatory protein α (SIRPα) fusion polypeptides of the disclosure may be used for the treatment of, for example, cardiovascular diseases, fibrosis, cancers, infectious diseases, hematological diseases, and neurological diseases.

### I. Definitions

Unless otherwise defined herein, scientific and technical terms used herein shall have the meanings that are commonly understood by those of ordinary skill in the art. Generally, nomenclature used in connection with, and techniques of, chemistry, molecular biology, cell biology, immunology, pharmacology, and protein chemistry, described herein, are those well-known and commonly used in the art.

As used herein and in the appended claims, the singular forms “a,” “and,” and “the” include plural referents unless the context clearly dictates otherwise. Thus, for example, reference to “an agent” refers to one or mixtures of such candidates, and reference to “a method” includes reference to equivalent steps and methods known to those skilled in the art, and so forth.

As used herein, the term “approximately” or “about,” as applied to one or more values of interest, refers to a value that is similar in magnitude and/or within a similar range to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the disclosure, subject to any specifically excluded limit in the stated range. Where the stated range includes one or both of the limits, ranges excluding either or both of those included limits are also included in the disclosure.

As used herein, the terms “polypeptide,” “peptide,” and “protein” refer to polymers of amino acids of any length. The terms also encompass an amino acid polymer that has been modified; for example, to include disulfide bond formation, glycosylation, lipidation, phosphorylation, or conjugation with a labeling component.

As used herein, the term “nucleic acid sequence” or “nucleotide sequence” refers to a molecule comprising either of a sequence of DNA or RNA nucleotides, presented from 5′ to 3′.

As used herein, “antibody” includes reference to a full-length immunoglobulin molecule immunologically reactive with a particular antigen, including both polyclonal and monoclonal antibodies. The term includes humanized antibodies, chimeric antibodies e.g., murine variable region with a human constant region, and conjugated antibodies.

The term “Fc domain” (also interchangeably referred to herein as “Fc sequence”, “Fc region”, or simply as “Fc”) as used herein refers to a fragment crystallizable region monomer of an antibody domain comprising a constant heavy chain 2 domain (CH2) and a constant heavy chain 3 domain (CH3). In some embodiments the “Fc domain” sequence comprises a IgG hinge region sequence. In some embodiments, Fc domains dimerize or form other multimers. Exemplary human Fc domains include IgG1, IgG2, IgG3, and IgG4 Fc domains.

Unless otherwise noted, modifications in an Fc domain are presented according to the EU numbering scheme. However, there are multiple numbering schemes which can be easily cross-referenced by one of skill in the art (www.imgt.org/IMGTScientificChart/Numbering/Hu_IGHGnber.html#refs).

The terms “treatment”, “treating” and the like are used herein to generally mean obtaining a desired pharmacologic and/or physiologic effect with a therapeutic agent. The effect may be prophylactic in terms of completely or partially preventing a disease or symptom thereof, e.g., reducing the likelihood that the disease or symptom thereof occurs in the subject, and/or may be therapeutic in terms of completely or partially reducing a symptom, or a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting or slowing the onset or development of the disease; or (c) relieving the disease, e.g., causing regression of the disease or symptoms associated with the disease. The therapeutic agent may be administered before, during or after the onset of disease. The treatment of ongoing disease, where the treatment stabilizes or reduces the undesirable clinical symptoms of the patient, may be of particular interest. In some embodiments, treatment is performed prior to complete loss of function in the affected tissues. In some embodiments, the subject's treatment will be administered during the symptomatic stage of the disease, and in some embodiments, after the symptomatic stage of the disease.

The terms “individual,” “subject,” and “patient” are used interchangeably herein and refer to any subject for whom treatment is desired. The subject may be a mammalian subject. Mammalian subjects include, e. g., humans, non-human primates, rodents, (e.g., rats, mice), lagomorphs (e.g., rabbits), ungulates (e.g., cows, sheep, pigs, horses, goats, and the like), etc. In some embodiments, the subject is a human. In some embodiments, the subject is a non-human primate, for example a cynomolgus monkey. In some embodiments, the subject is a companion animal (e.g., cats, dogs).

### II. Signal-Regulatory Protein α (SIRPα) Fusion Polypeptides

The blocking of endogenous signal-regulatory protein α (SIRPα) binding to a CD47 protein on cells, e.g., on human cells, allows for phagocytic engulfment of a CD47-expressing cell. Provided herein are fusion polypeptides comprising a SIRPα domain and a modified Fc domain, useful for the blocking endogenous SIRPα binding to a CD47 protein, wherein the inclusion of the modified Fc domain improves the pharmacokinetics and/or pharmacodynamics of the fusion polypeptide. In some embodiments, the inclusion of the modified Fc domain, among other effects, can increase the binding affinity of the fusion polypeptide to a neonatal Fc receptor, thereby increasing the half-life of the fusion polypeptide in the subject, and leading to improved blocking of the binding of endogenous SIRPα to CD47 by the fusion polypeptide. In some embodiments, the inclusion of the modified Fc domain, among other effects, can increase the binding affinity of the fusion polypeptide to a CD47 protein. In some embodiments such increase in binding affinity allows for decreasing the dose and/or dosage frequency of the fusion polypeptide in the subject, and may lead to improvements in the blocking of the binding of endogenous SIRPα to CD47 by the fusion polypeptide. In some embodiments, the inclusion of the modified Fc domain, among other effects, can increase the safety of the SIRPα fusion polypeptide.

In some embodiments, a SIRPα fusion polypeptide of the disclosure forms a monomer, dimer, trimer, tetramer, pentamer, or other multimer. In exemplary embodiments, a SIRPα fusion polypeptide comprising a modified Fc domain of the disclosure forms a dimer, in some embodiments the dimer is a homodimer, whereas in other embodiments the dimer is a heterodimer.

### SIRPα Polypeptide Sequences

A SIRPα fusion polypeptide as provided herein comprises a SIRPα domain and a modified Fc domain. In this section, the SIRPα domain aspect of the fusion polypeptide is described in greater detail.

The SIRPα domains provided herein comprise a membrane distal (D1) domain of SIRPα, which binds to the CD47 protein (either wild type of modified versions thereof). In some embodiments, a SIRPα fusion polypeptide of the disclosure comprises a wild type human SIRPα D1 sequence comprising SEQ ID NO: 1 or an amino acid sequence with at least 70%, at least 75%, at least 80%, at least 85%, at least 90%, at least 95%, at least 96%, at least 97%, at least 98%, or at least 99% sequence identity thereto.

SEQ ID NO: 1
EEELQVIQPDKSVSVAAGESAILHCTVTSLIPVGPIQWFRGAGPARELI
YNQKEGHFPRVTTVSESTKRENMDFSISISNITPADAGTYYCVKFRKGS
PDTEFKSGAGTELSVRAKPS

In some embodiments, a SIRPα fusion polypeptide of the disclosure comprise a SIRPα D1 domain with one or more amino acid modifications relative to a wild type sequence of the D1 domain, for example a D1 domain of SEQ ID NO: 1. A modification includes an amino acid substitution, an amino acid deletion, and an amino acid addition. In some embodiments, a SIRPα fusion polypeptide comprises a SIRPα D1 domain, with at least one amino acid modification relative to the wild-type sequence of the D1 domain. In some embodiments, a SIRPα fusion polypeptide comprises a SIRPα D1 domain, with at least two amino acid modifications relative to the wild-type sequence of the D1 domain. In some embodiments, a SIRPα fusion polypeptide comprises a SIRPα D1 domain, with at least three amino acid modifications relative to the wild-type sequence of the D1 domain. In some embodiments, a SIRPα fusion polypeptide comprises a SIRPα D1 domain, with at least four amino acid modifications relative to the wild-type sequence of the D1 domain. In some embodiments, a SIRPα fusion polypeptide comprises a SIRPα D1 domain, with at least five amino acid modifications relative to the wild-type sequence of the D1 domain. In some embodiments, a SIRPα fusion polypeptide comprises a SIRPα D1 domain, with at least six amino acid modifications relative to the wild-type sequence of the D1 domain. In some embodiments, a SIRPα fusion polypeptide comprises a SIRPα D1 domain, with at least seven amino acid modifications relative to the wild-type sequence of the D1 domain. In some embodiments, a SIRPα fusion polypeptide comprises a SIRPα D1 domain, with at least eight amino acid modifications relative to the wild-type sequence of the D1 domain. In some embodiments, a SIRPα fusion polypeptide comprises a SIRPα D1 domain, with at least nine amino acid modifications relative to the wild-type sequence of the D1 domain. In some embodiments, a SIRPα fusion polypeptide comprises a SIRPα D1 domain, with at least ten amino acid modifications relative to the wild-type sequence of the D1 domain. In some embodiments, a SIRPα fusion polypeptide comprises a SIRPα D1 domain, with at least eleven amino acid modifications relative to the wild-type sequence of the D1 domain. In some embodiments, a SIRPα fusion polypeptide comprises a SIRPα D1 domain, with at least twelve amino acid modifications relative to the wild-type sequence of the D1 domain. In some embodiments, a SIRPα fusion polypeptide comprises a SIRPα D1 domain, with at least thirteen amino acid modifications relative to the wild-type sequence of the D1 domain. In some embodiments, a SIRPα fusion polypeptide comprises a SIRPα D1 domain, with at least fourteen amino acid modifications relative to the wild-type sequence of the D1 domain. In some embodiments, a SIRPα fusion polypeptide comprises a SIRPα D1 domain, with at least fifteen amino acid modifications relative to the wild-type sequence of the D1 domain. In some embodiments, a SIRPα fusion polypeptide comprises a SIRPα D1 domain, with at least sixteen amino acid modifications relative to the wild-type sequence of the D1 domain. In some embodiments, a SIRPα fusion polypeptide comprises a SIRPα D1 domain, with at least seventeen amino acid modifications relative to the wild-type sequence of the D1 domain. In some embodiments, a SIRPα fusion polypeptide comprises a SIRPα D1 domain, with at least eighteen amino acid modifications relative to the wild-type sequence of the D1 domain. In some embodiments, a SIRPα fusion polypeptide comprises a SIRPα D1 domain, with at least nineteen amino acid modifications relative to the wild-type sequence of the D1 domain. In some embodiments, a SIRPα fusion polypeptide comprises a SIRPα D1 domain, with at least twenty amino acid modifications relative to the wild-type sequence of the D1 domain. In some embodiments, a SIRPα fusion polypeptide comprises a SIRPα D1 domain, with at least twenty-one amino acid modifications relative to the wild-type sequence of the D1 domain. In some embodiments, a SIRPα fusion polypeptide comprises a SIRPα D1 domain, with at least twenty-two amino acid modifications relative to the wild-type sequence of the D1 domain. In some embodiments, a SIRPα fusion polypeptide comprises a SIRPα D1 domain, with at least twenty-three amino acid modifications relative to the wild-type sequence of the D1 domain. In some embodiments, a SIRPα fusion polypeptide comprises a SIRPα D1 domain, with at least twenty-four amino acid modifications relative to the wild-type sequence of the D1 domain. In some embodiments, a SIRPα fusion polypeptide comprises a SIRPα D1 domain, with at least twenty-five amino acid modifications relative to the wild-type sequence of the D1 domain.

In some embodiments, a SIRPα fusion polypeptide comprises a SIRPα D1 polypeptide that exhibits a higher binding affinity (i.e., lower K_{D} value) to CD47 by at least 5-fold, 10-fold, at least 20-fold, at least 50-fold, at least 100-fold, at least 500-fold, at least 1000-fold or more relative to a wild type SIRPα D1 domain.

In some embodiments, a SIRPα fusion polypeptide of the disclosure comprises a modification relative to the wild type SIRPα D1 domain sequence of SEQ ID NO: 1 at one or more of the following residues V6, S14, S20, I22, H24, V27, I31, A45, E47, K53, E54, H56, S66, E70, S77, V92, and/or a duplication of the D100 residue.

In some embodiments, a SIRPα fusion polypeptide of the disclosure comprises a modification relative to the wild type SIRPα D1 domain sequence of SEQ ID NO: 1 of one or more of the following: V6I, S14L, S20T, I22T, H24R, V27I, I31F, A45G, E47V, K53R, E54Q, H56P, S66T, E70N, S77R, V92I, and/or a duplication of the D100 residue.

In some embodiments, a SIRPα fusion polypeptide of the disclosure comprises a SIRPα D1 sequence of SEQ ID NO: 2 (referred to herein as CV1, an exemplary SIRPα D1 domain which exhibits higher binding affinity to CD47), or an amino acid sequence with at least 70%, at least 75%, at least 80%, at least 85%, at least 90%, at least 95%, at least 96%, at least 97%, at least 98%, or at least 99% sequence identity thereto.

SEQ ID NO: 2
EEELQIIQPDKSVLVAAGETATLRCTITSLFPVGPIQWFRGAGPGRVLI
YNQRQGPFPRVTTVSDTTKRNNMDFSIRIGNITPADAGTYYCIKFRKGS
PDDVEFKSGAGTELSVRAKPS

In some embodiments, a SIRPα fusion polypeptide of the disclosure comprises a SIRPα D1 sequence of SEQ ID NO: 3 or an amino acid sequence with at least 70%, at least 75%, at least 80%, at least 85%, at least 90%, at least 95%, at least 96%, at least 97%, at least 98%, or at least 99% sequence identity thereto.

SEQ ID NO: 3
XXELQVIQPDKSVLVAAGETATLRCTATSLIPVGPIQWFRGAGPGRELI
YNQKEGHFPRVTTVSDLTKRNNMDFSIRIGNITPADAGTYYCVKFRKGS
PDDVEFKSGAGTELSVR

In some embodiments, a SIRPα fusion polypeptide of the disclosure comprises a SIRPα D1 sequence of SEQ ID NO: 4 or an amino acid sequence with at least 70%, at least 75%, at least 80%, at least 85%, at least 90%, at least 95%, at least 96%, at least 97%, at least 98%, or at least 99% sequence identity thereto.

SEQ ID NO: 4
XXELQVIQPDKSVSVAAGESAILHCTVTSLIPVGPIQWFRGAGPARELI
YNQKEGHFPRVTTVSESTKRENMDFSISISNITPADAGTYYCVKFRKGS
PDTEFKSGAGTELSVR

In some embodiments, a SIRPα fusion polypeptide of the disclosure comprises a SIRPα D1 sequence of SEQ ID NO: 5 or an amino acid sequence with at least 70%, at least 75%, at least 80%, at least 85%, at least 90%, at least 95%, at least 96%, at least 97%, at least 98%, or at least 99% sequence identity thereto.

SEQ ID NO: 5
EEXLQVIQPDKXVXVAAGEXAXLXCTXTSLIPVGPIQWFRGAGPXRELI
YNQKEGHFPRVTTVSXXDLTKRXNMDFXIXIXNITPADAGTYYCVKFRK
GSPDDXEFKSGAGTELSVR

In some embodiments, a SIRPα fusion polypeptide of the disclosure comprises one or more of the following substitutions relative to the SIRPα D1 sequences of SEQ ID NOS: 1-5: E3G, L4V, L4I, V6I, V6L, S12F, S14L, S20T, A21V, I22T, H24L, H24R, V27A, V27I, V27L, I31F, I31S, I31T, Q37H, A45G, E47V, E47L, K53R, E54Q, E54P, H56P, H56R, V63I, E65D, S66T, S66G, S66L, K68R, E70N, M72R, S75P, R77S, S79G, N80A, N80X, I81N, T82N, P83N, P83X, V92I, F94L, F94V, duplication of D100, E102V, E102T, E102F, F103E, F103V, K104F, K104V, A115G, K116A, and K116G, wherein X=any amino acid.

In some embodiments, a SIRPα fusion polypeptide sequence of the disclosure may comprise any of the SIRPα D1 sequences described in WO2013109752, WO2014094122A1, WO2017027422, WO2016023040, and WO2016024021A1, incorporated herein in their entirety.

### III. Modified Fc Domains for Improved SIRPα Fusion Polypeptide Pharmacokinetics

The present disclosure provides signal-regulatory protein α (SIRPα) fusion polypeptides comprising modified Fc domains, useful for improved pharmacokinetics and/or pharmacodynamics e.g., by increasing the binding affinity to FcRn and/or to the CD47 protein. As provided herein, one or more modifications (e.g., substitutions) may be introduced into a wild type IgG Fc domain sequence, e.g., a human IgG1, IgG2, IgG3, or IgG4 domain to increase engagement with the neonatal Fc receptor (FcRn) and extend the half-life of the fusion polypeptide. The one or more modifications introduced into a wild type IgG Fc domain sequence may also improve the efficacy and/or safety of the fusion polypeptide.

### Modified Fc Domain Sequences

The SIRPα fusion polypeptides provided herein comprise modified Fc domains, useful for improved pharmacokinetics and/or pharmacodynamics of the fusion polypeptide. The Fc domains provided herein can be modified domains of any species, e.g., human or mouse, or may be an engineered non-naturally occurring Fc domain, e.g. a human or mouse IgG domain comprising one or more modifications. In some embodiments, the Fc domain is a modified human IgG1 or IgG4 Fc domain. Canonical wild type sequences for these are presented herein.

In some embodiments the Fc domain is a modified human IgG2 or IgG3 domain, or a mouse IgG1, IgG2a, IgG2b, or IgG3 domain.

In some embodiments, a SIRPα fusion polypeptide of the disclosure comprises the human IgG1 Fc amino sequence of SEQ ID NO: 6 or an amino acid sequence with at least 70%, at least 75%, at least 80%, at least 85%, at least 90%, at least 95%, at least 96%, at least 97%, at least 98%, or at least 99% sequence identity thereto.

SEQ ID NO: 6
ELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPA
PIEKTISKAKGQPREPQVYTLPPSRDELTKNQVSLTCLVKGFYPSDIAV
EWESNGQPENNYKTTPPVLDSDGSFFLYSKLTVDKSRWQQGNVFSCSVM
HEALHNHYTQKSLSLSPGK

In some embodiments, a SIRPα fusion polypeptide of the disclosure comprises the human IgG4 Fc amino sequence of SEQ ID NO: 7 or an amino acid sequence with at least 70%, at least 75%, at least 80%, at least 85%, at least 90%, at least 95%, at least 96%, at least 97%, at least 98%, or at least 99% sequence identity thereto.

SEQ ID NO: 7
PPCPSCPAPEFLGGPSVFLFPPKPKDTLMISRTPEVTCVVVDVSQEDPE
VQFNWYVDGVEVHNAKTKPREEQFNSTYRVVSVLTVLHQDWLNGKEYKC
KVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQE
GNVFSCSVMHEALHNHYTQKSLSLSLGK

In some embodiments, a SIRPα fusion polypeptide of the disclosure comprises an Fc domain of an IgG4 human Fc domain and the polypeptide is prone to the dynamic process of Fab-arm exchange. Accordingly, in some embodiments the IgG4 Fc domain may comprise a S228P substitution relative to SEQ ID NO: 7 according to EU numbering scheme, resulting in the reduction of this process. In some embodiments, a SIRPα fusion polypeptide of the disclosure comprises the IgG4 Fc amino sequence of SEQ ID NO: 8 or an amino acid sequence with at least 70%, at least 75%, at least 80%, at least 85%, at least 90%, at least 95%, at least 96%, at least 97%, at least 98%, or at least 99% sequence identity thereto.

SEQ ID NO: 8
PPCPPCPAPEFLGGPSVFLFPPKPKDTLMISRTPEVTCVVVDVSQEDPE
VQFNWYVDGVEVHNAKTKPREEQFNSTYRVVSVLTVLHQDWLNGKEYKC
KVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQE
GNVFSCSVMHEALHNHYTQKSLSLSLGK

In some embodiments, the IgG4Fc amino acid sequence comprises the substitution L445P relative to SEQ ID NO: 8, according to the EU numbering scheme.

In some embodiments, a SIRPα fusion polypeptide of the disclosure comprises the human IgG1 Fc sequence of SEQ ID NO: 6 comprising one or more modifications (e.g., substitutions) to increase effector function. In some embodiments, the substitutions are selected from the group consisting of V215A, G236A, S239D, I332E, T250Q, M252Y, S254T, T256E, S267E, N325S, L328F, N343S, M428L, H433K, and N434F relative to SEQ ID NO: 6 according to the EU numbering scheme. Exemplary combinations include: G236A-S239D, G236A-I332E, S239D-I332E, V215A-G236A-S239D-I332E, G236A-S239D-I332E, K326W-E333S, S267E-H268F-S324T, and E345R-E430G-S440Y, F243L-R292P-Y300L-V305I -P396L, S239D-I332E, S298A-E333A-K334A, L234Y-L235Q-G236W-S239M-H268D-D270E-S298A, and D270E-K326D-A330M-K334E, relative to SEQ ID NO: 6 according to the EU numbering scheme.

In some embodiments, a SIRPα fusion polypeptide of the disclosure comprises a human IgG1Fc sequence of SEQ ID NO: 6 comprising one or more modifications (e.g., substitutions) to decrease effector function. In some embodiments, the substitutions are selected from the group consisting of: N297A, N297Q, N297G, L235E, L234A, L235A, K214R, P329G, D356E, and L358M.

In some embodiments, a SIRPα fusion polypeptide of the disclosure comprises a human IgG4 Fc sequence of SEQ ID NOS: 7 or 8 comprising one or more modifications (e.g., substitutions) to decrease effector function. In some embodiments, the substitutions are selected from the group consisting of: L235A, L235E, S228P, and F234A. Exemplary combinations include L235E-S228P, S228P-F234A, and S228P-F234A-L235A.

In other embodiments, a SIRPα fusion polypeptide of the disclosure comprises an IgG1 Fc or an IgG4 Fc domain in which a modification is present to increase serum half-life. In some embodiments the mutations are selected from the group consisting of T250Q, M252Y, S254T, T256E, S267E, N325S, L328F, N343S, M428L, N434F, and H443K relative to IgG1 Fc domain sequence SEQ ID NO: 6 or IgG4 Fc domain sequence SEQ ID NOS: 7 or 8 according to the EU numbering scheme. In some embodiments the mutations are selected from the group consisting of T250Q-M428L, M252Y-S254T-T256E, M428L-N434S, S267E-L328F, N325S-L328F, and H433K-N434F, relative to IgG1 Fc domain sequence SEQ ID NO: 6 or IgG4 Fc domain sequence SEQ ID NOS: 7 or 8 according to the EU numbering scheme.

In embodiments, an exemplary SIRPα fusion polypeptide of the disclosure comprises an IgG4 human Fc domain of SEQ ID NO: 8 comprising substitutions M252Y, S254T, and T256E, relative to SEQ ID NO: 8

In some embodiments, a SIRPα fusion polypeptide of the disclosure comprises the human IgG4 Fc amino sequence of SEQ ID NO: 9 or an amino acid sequence with at least 70%, at least 75%, at least 80%, at least 85%, at least 90%, at least 95%, at least 96%, at least 97%, at least 98%, or at least 99% sequence identity thereto.

SEQ ID NO: 9
PPCPPCPAPEFLGGPSVFLFPPKPKDTLYITREPEVTCVVVDVSQEDPE
VQFNWYVDGVEVHNAKTKPREEQFNSTYRVVSVLTVLHQDWLNGKEYKC
KVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQE
GNVFSCSVMHEALHNHYTQKSLSLSLGK

In some embodiments, a SIRPα fusion polypeptide of the disclosure comprises a peptide linker joining the SIRPα domain and the Fc domain. In some embodiments, the SIRPα fusion polypeptide comprises a peptide linker of about 6, about 7, about 8, about 9, about 10, about 11, about 12, about 13, about 14, about 15, or about 16 amino acids in length. In some embodiments, the peptide linker comprises Alanine (A), Glycine (G) and/or Serine (S) amino acids. In some embodiments, the peptide linker is 8 amino acids of G and S amino acids. In some embodiments, the linker is AAA. In some embodiments, the linker is GGGSGGGS (SEQ ID NO: 11). In some embodiments, the linker comprises a human IgG sequence, e.g., ASTKGPSVFPLAP (SEQ ID NO: 12).

In exemplary embodiments, a SIRPα fusion polypeptide as provided herein comprises a SIRPα domain sequence of any of SEQ ID NOS: 1-5 or an amino acid sequence with at least 70%, at least 75%, at least 80%, at least 85%, at least 90%, at least 95%, at least 96%, at least 97%, at least 98%, or at least 99% sequence identity thereto; and a modified Fc domain sequence of any of SEQ ID NOS: 6-9 or an amino acid sequence with at least 70%, at least 75%, at least 80%, at least 85%, at least 90%, at least 95%, at least 96%, at least 97%, at least 98%, or at least 99% sequence identity thereto.

In exemplary embodiments, a SIRPα fusion polypeptide as provided herein comprises the Fc domain sequence of SEQ ID NO: 9 or an amino acid sequence with at least 70%, at least 75%, at least 80%, at least 85%, at least 90%, at least 95%, at least 96%, at least 97%, at least 98%, or at least 99% sequence identity thereto; and the SIRPα domain sequence of SEQ ID NO: 2 or an amino acid sequence with at least 70%, at least 75%, at least 80%, at least 85%, at least 90%, at least 95%, at least 96%, at least 97%, at least 98%, or at least 99% sequence identity thereto.

Exemplary SIRPα fusion polypeptides comprising a SIRPα domain and a modified Fc domain comprise the sequence of SEQ ID NO: 10, or an amino acid sequence with at least 70%, at least 75%, at least 80%, at least 85%, at least 90%, at least 95%, at least 96%, at least 97%, at least 98%, or at least 99% sequence identity thereto.

SEQ ID NO: 10
VTTVSDTTKRNNMDFSIRIGNITPADAGTYYCIKFRKGSPDDVEFKSGA
GTELSVRAKPSAAAPPCPPCPAPEFLGGPSVFLFPPKPKDTLYITREPE
VTCVVVDVSQEDPEVQFNWYVDGVEVHNAKTKPREEQFNSTYRVVSVLT
VLHQDWLNGKEYKCKVSNKGLPSSIEKTISKAKGQPREPQVYTLPPSQE
EMTKNQVSLTCLVKGFYPSDIAVEWESNGQPENNYKTTPPVLDSDGSFF
LYSRLTVDKSRWQEGNVFSCSVMHEALHNHYTQKSLSLSLGK

### Modified Fc Domain Effects

In some embodiments, one or more modifications may be introduced in an Fc domain of a SIRPα fusion polypeptide of the disclosure to increase binding affinity of the fusion polypeptide to FcRn and/or to increase binding affinity to a CD47 protein. In some embodiments, inclusion of the modified Fc domain leads to an increase in binding affinity to FcRn by about 1.5×, about 2×, about 3×, about 4×, about 5×, about 6×, about 7×, about 8×, about 9×, about 10×, about 20×, about 50×, about 100×, about 1000×, or about 10,000× relative to that of a wild type Fc domain. In some embodiments, inclusion of the modified Fc domain leads to an increase in binding affinity to CD47 by about 1.5×, about 2×, about 3×, about 4×, about 5×, about 6×, about 7×, about 8×, about 9×, about 10×, about 20×, about 50×, about 100×, about 250×, about 500×, about 750×, about 1000×, about 5000× or about 10,000× relative to that of a wild type Fc domain. In some embodiments, the CD47 protein is a human, mouse, non-human primate, or a rat CD47 protein. In some embodiments the one or more modification in the Fc domain is selected from the group consisting of T250Q-M428L, M252Y-S254T-T256E, M428L-N434S, S267E-L328F, N325S-L328F, and H433K-N434F, relative to IgG1 Fc domain sequence SEQ ID NO: 6 or IgG4 Fc domain sequence SEQ ID NOS: 7 or 8 according to the EU numbering scheme. In embodiments, an exemplary SIRPα fusion polypeptide of the disclosure comprises an IgG4 human Fc domain of SEQ ID NO: 8 comprising substitutions M252Y, S254T, and T256E, relative to SEQ ID NO: 8. Exemplary SIRPα fusion polypeptides of the disclosure comprise the sequence of SEQ ID NO: 10, or an amino acid sequence with at least 70%, at least 75%, at least 80%, at least 85%, at least 90%, at least 95%, at least 96%, at least 97%, at least 98%, or at least 99% sequence identity thereto.

In some embodiments, inclusion of the modified Fc domain increases the half-life, in vivo, or in vitro, of the SIRPα fusion polypeptide.

In some embodiments, the SIRPα fusion polypeptide comprising the modified Fc domain exhibits an increase in the in vivo half life by about 1 about 1.5×, about 2×, about 3×, about 4×, about 5×, about 6×, about 7×, about 8×, about 9×, about 10×, about 20×, about 50×, about 100×, about 250×, about 500×, about 750×, about 1000×, about 5000× or about 10,000×, relative to the half life of a SIRPα fusion polypeptide comprising a wild type Fc domain. In some embodiments the one or more modification in the Fc domain is selected from the group consisting of T250Q-M428L, M252Y-S254T-T256E, M428L-N434S, S267E-L328F, N325S-L328F, and H433K-N434F, relative to IgG1 Fc domain sequence SEQ ID NO: 6 or IgG4 Fc domain sequence SEQ ID NOS: 7 or 8 according to the EU numbering scheme. In embodiments, an exemplary SIRPα fusion polypeptide of the disclosure comprises an IgG4 human Fc domain of SEQ ID NO: 8 comprising substitutions M252Y, S254T, and T256E, relative to SEQ ID NO: 8. Exemplary SIRPα fusion polypeptides of the disclosure comprise the sequence of SEQ ID NO: 10, or an amino acid sequence with at least 70%, at least 75%, at least 80%, at least 85%, at least 90%, at least 95%, at least 96%, at least 97%, at least 98%, or at least 99% sequence identity thereto.

In some embodiments, inclusion of the modified Fc domain increases the in vitro (e.g. cell culture) half life of the SIRPα fusion polypeptide by about 1.5×, about 2×, about 3×, about 4×, about 5×, about 6×, about 7×, about 8×, about 9×, about 10×, about 20×, about 50×, about 100×, about 250×, about 500×, about 750×, about 1000×, about 5000× or about 10,000×, relative to the half life of a SIRPα fusion polypeptide comprising a wild type Fc domain. In some embodiments the one or more modification in the Fc domain is selected from the group consisting of T250Q-M428L, M252Y-S254T-T256E, M428L-N434S, S267E-L328F, N325S-L328F, and H433K-N434F, relative to IgG1 Fc domain sequence SEQ ID NO: 6 or IgG4 Fc domain sequence SEQ ID NOS: 7 or 8 according to the EU numbering scheme. In embodiments, an exemplary SIRPα fusion polypeptide of the disclosure comprises an IgG4 human Fc domain of SEQ ID NO: 8 comprising substitutions M252Y, S254T, and T256E, relative to SEQ ID NO: 8. Exemplary SIRPα fusion polypeptides of the disclosure comprise the sequence of SEQ ID NO: 10, or an amino acid sequence with at least 70%, at least 75%, at least 80%, at least 85%, at least 90%, at least 95%, at least 96%, at least 97%, at least 98%, or at least 99% sequence identity thereto.

In some embodiments, inclusion of a modified Fc domain slows the blood clearance of a SIRPα fusion polypeptide in a subject. In some embodiments, inclusion of a modified Fc domain slows the blood clearance of a SIRPα fusion polypeptide by about 1.5×, about 2×, about 3×, about 4×, about 5×, about 6×, about 7×, about 8×, about 9×, about 10×, about 20×, about 50×, about 100×, or about 1000×, relative to a SIRPα fusion polypeptide comprising a wild type Fc domain. In some embodiments the one or more modification in the Fc domain is selected from the group consisting of T250Q-M428L, M252Y-S254T-T256E, M428L-N434S, S267E-328F, N325S-L328F, and H433K-N434F, relative to IgG1 Fc domain sequence SEQ ID NO: 6 or IgG4 Fc domain sequence SEQ ID NOS: 7 or 8 according to the EU numbering scheme. In embodiments, an exemplary SIRPα fusion polypeptide of the disclosure comprises an IgG4 human Fc domain of SEQ ID NO: 8 comprising substitutions M252Y, S254T, and T256E, relative to SEQ ID NO: 8. Exemplary SIRPα fusion polypeptides of the disclosure comprise the sequence of SEQ ID NO: 10, or an amino acid sequence with at least 70%, at least 75%, at least 80%, at least 85%, at least 90%, at least 95%, at least 96%, at least 97%, at least 98%, or at least 99% sequence identity thereto.

In some embodiments, inclusion of the modified Fc domain increases the binding affinity in vivo, or in vitro, of the SIRPα fusion polypeptide to a CD47 protein. In some embodiments, the SIRPα fusion polypeptide comprising the modified Fc domain is effective at a lower dose in a subject relative to a SIRPα fusion polypeptide comprising a wild type Fc domain. In some embodiments the one or more modification in the Fc domain is selected from the group consisting of T250Q-M428L, M252Y-S254T-T256E, M428L-N434S, S267E-L328F, N325S-L328F, and H433K-N434F, relative to IgG1 Fc domain sequence SEQ ID NO: 6 or IgG4 Fc domain sequence SEQ ID NOS: 7 or 8 according to the EU numbering scheme. In embodiments, an exemplary SIRPα fusion polypeptide of the disclosure comprises an IgG4 human Fc domain of SEQ ID NO: 8 comprising substitutions M252Y, S254T, and T256E, relative to SEQ ID NO: 8. Exemplary SIRPα fusion polypeptides of the disclosure comprise the sequence of SEQ ID NO: 10, or an amino acid sequence with at least 70%, at least 75%, at least 80%, at least 85%, at least 90%, at least 95%, at least 96%, at least 97%, at least 98%, or at least 99% sequence identity thereto.

In some embodiments, inclusion of a modified Fc domain reduces the EC₅₀ of a SIRPα fusion polypeptide by about about 1.5×, about 2×, about 3×, about 4×, about 5×, about 6×, about 7×, about 8×, about 9×, about 10×, about 20×, about 50×, about 100×, about 250×, about 500×, about 750×, about 1000×, about 5000× or about 10,000×, relative to a SIRPα fusion polypeptide comprising a wild type Fc domain. In some embodiments the one or more modification in the Fc domain is selected from the group consisting of T250Q-M428L, M252Y-S254T-T256E, M428L-N434S, S267E-L328F, N325S-L328F, and H433K-N434F, relative to IgG1 Fc domain sequence SEQ ID NO: 6 or IgG4 Fc domain sequence SEQ ID NOS: 7 or 8 according to the EU numbering scheme. In embodiments, an exemplary SIRPα fusion polypeptide of the disclosure comprises an IgG4 human Fc domain of SEQ ID NO: 8 comprising substitutions M252Y, S254T, and T256E, relative to SEQ ID NO: 8. Exemplary SIRPα fusion polypeptides of the disclosure comprise the sequence of SEQ ID NO: 10, or an amino acid sequence with at least 70%, at least 75%, at least 80%, at least 85%, at least 90%, at least 95%, at least 96%, at least 97%, at least 98%, or at least 99% sequence identity thereto.

In some embodiments, inclusion of a modified Fc domain reduces the effective dose of a SIRPα fusion polypeptide necessary to achieve a desired therapeutic effect by about about 1.5×, about 2×, about 3×, about 4×, about 5×, about 6×, about 7×, about 8×, about 9×, about 10×, about 20×, about 50×, about 100×, about 250×, about 500×, about 750×, about 1000×, about 5000× or about 10,000×, relative to a SIRPα fusion polypeptide comprising a wild type Fc domain. In some embodiments the one or more modification in the Fc domain is selected from the group consisting of T250Q-M428L, M252Y-S254T-T256E, M428L-N434S, S267E-L328F, N325S-L328F, and H433K-N434F, relative to IgG1 Fc domain sequence SEQ ID NO: 6 or IgG4 Fc domain sequence SEQ ID NOS: 7 or 8 according to the EU numbering scheme. In embodiments, an exemplary SIRPα fusion polypeptide of the disclosure comprises an IgG4 human Fc domain of SEQ ID NO: 8 comprising substitutions M252Y, S254T, and T256E, relative to SEQ ID NO: 8. Exemplary SIRPα fusion polypeptides of the disclosure comprise the sequence of SEQ ID NO: 10, or an amino acid sequence with at least 70%, at least 75%, at least 80%, at least 85%, at least 90%, at least 95%, at least 96%, at least 97%, at least 98%, or at least 99% sequence identity thereto.

In some embodiments, the SIRPα fusion polypeptide comprising the modified Fc domain is effective at a lower dosage frequency (necessary to achieve a desired therapeutic effect) in a subject relative to a SIRPα fusion polypeptide comprising a wild type Fc domain. In some embodiments the one or more modification in the Fc domain is selected from the group consisting of T250Q-M428L, M252Y-S254T-T256E, M428L-N434S, S267E-L328F, N325S-L328F, and H433K-N434F, relative to IgG1 Fc domain sequence SEQ ID NO: 6 or IgG4 Fc domain sequence SEQ ID NOS: 7 or 8 according to the EU numbering scheme. In embodiments, an exemplary SIRPα fusion polypeptide of the disclosure comprises an IgG4 human Fc domain of SEQ ID NO: 8 comprising substitutions M252Y, S254T, and T256E, relative to SEQ ID NO: 8. Exemplary SIRPα fusion polypeptides of the disclosure comprise the sequence of SEQ ID NO: 10, or an amino acid sequence with at least 70%, at least 75%, at least 80%, at least 85%, at least 90%, at least 95%, at least 96%, at least 97%, at least 98%, or at least 99% sequence identity thereto.

In some embodiments, inclusion of a modified Fc domain reduces the dosage frequency of a SIRPα fusion polypeptide necessary to achieve a desired therapeutic effect by about 1.5×, about 2×, about 3×, about 4×, about 5×, about 6×, about 7×, about 8×, about 9×, about 10×, about 20×, about 50×, about 100×, about 250×, about 500×, about 750×, about 1000×, about 5000× or about 10,000×, relative to a SIRPα fusion polypeptide comprising a wild type Fc domain. In some embodiments the one or more modification in the Fc domain is selected from the group consisting of T250Q-M428L, M252Y-S254T-T256E, M428L-N434S, S267E-L328F, N325S-L328F, and H433K-N434F, relative to an IgG1 Fc domain sequence of SEQ ID NO: 6 or an IgG4 Fc domain sequence of SEQ ID NOS: 7 or 8, according to the EU numbering scheme. In embodiments, an exemplary SIRPα fusion polypeptide of the disclosure comprises an IgG4 human Fc domain of SEQ ID NO: 8 comprising substitutions M252Y, S254T, and T256E, relative to SEQ ID NO: 8. Other exemplary SIRPα fusion polypeptides of the disclosure comprise the sequence of SEQ ID NO: 10, or an amino acid sequence with at least 70%, at least 75%, at least 80%, at least 85%, at least 90%, at least 95%, at least 96%, at least 97%, at least 98%, or at least 99% sequence identity thereto.

In some embodiments, the inclusion of a modified Fc domain increases the effector function of the Fc domain in a SIRPα fusion polypeptide. In some embodiments, inclusion of the modified Fc domain increases complement dependent cytotoxicity. In some embodiments, inclusion of the modified Fc domain increases complement dependent cytotoxicity of the SIRPα fusion polypeptide by about about 1.5×, about 2×, about 3×, about 4×, about 5×, about 6×, about 7×, about 8×, about 9×, about 10×, about 20×, about 50×, about 100×, about 250×, about 500×, about 750×, about 1000×, about 5000× or about 10,000×, relative to that of a SIRPα fusion polypeptide comprising a wild type Fc domain. In some embodiments the one or more modification in the Fc domain is selected from the group consisting of T250Q-M428L, M252Y-S254T-T256E, M428L-N434S, S267E-L328F, N325S-L328F, and H433K-N434F, relative to IgG1 Fc domain sequence SEQ ID NO: 6 or IgG4 Fc domain sequence SEQ ID NOS: 7 or 8 according to the EU numbering scheme. In embodiments, an exemplary SIRPα fusion polypeptide of the disclosure comprises an IgG4 human Fc domain of SEQ ID NO: 8 comprising substitutions M252Y, S254T, and T256E, relative to SEQ ID NO: 8. Exemplary SIRPα fusion polypeptides of the disclosure comprise the sequence of SEQ ID NO: 10, or an amino acid sequence with at least 70%, at least 75%, at least 80%, at least 85%, at least 90%, at least 95%, at least 96%, at least 97%, at least 98%, or at least 99% sequence identity thereto.

In some embodiments, inclusion of a modified Fc domain increases the interaction of the SIRPα fusion polypeptide with cells expressing a CD47 polypeptide. In some embodiments, inclusion of the modified Fc domain increases binding of the SIRPα fusion polypeptide to a CD47 expressing cell by about 1.5×, about 2×, about 3×, about 4×, about 5×, about 6×, about 7×, about 8×, about 9×, about 10×, about 20×, about 50×, about 100×, about 250×, about 500×, about 750×, about 1000×, about 5000× or about 10,000×, relative to that of a SIRPα fusion polypeptide comprising a wild type Fc domain. In some embodiments the one or more modification in the Fc domain is selected from the group consisting of T250Q-M428L, M252Y-S254T-T256E, M428L-N434S, S267E-L328F, N325S-L328F, and H433K-N434F, relative to IgG1 Fc domain sequence SEQ ID NO: 6 or IgG4 Fc domain sequence SEQ ID NOS: 7 or 8 according to the EU numbering scheme. In embodiments, an exemplary SIRPα fusion polypeptide of the disclosure comprises an IgG4 human Fc domain of SEQ ID NO: 8 comprising substitutions M252Y, S254T, and T256E, relative to SEQ ID NO: 8. Exemplary SIRPα fusion polypeptides of the disclosure comprise the sequence of SEQ ID NO: 10, or an amino acid sequence with at least 70%, at least 75%, at least 80%, at least 85%, at least 90%, at least 95%, at least 96%, at least 97%, at least 98%, or at least 99% sequence identity thereto.

In some embodiments, inclusion of a modified Fc domain increases SIRPα fusion polypeptide induction of endocytosis of a CD47 protein in cells expressing a CD47 polypeptide. In some embodiments, inclusion of the modified Fc domain increases SIRPα fusion polypeptide induction of endocytosis of a CD47 protein in a CD47 expressing cell by about 1.5×, about 2×, about 3×, about 4×, about 5×, about 6×, about 7×, about 8×, about 9×, about 10×, about 20×, about 50×, about 100×, about 250×, about 500×, about 750×, about 1000×, about 5000× or about 10,000×, relative to that of a SIRPα fusion polypeptide comprising a wild type Fc domain. In some embodiments the one or more modification in the Fc domain is selected from the group consisting of T250Q-M428L, M252Y-S254T-T256E, M428L-N434S, S267E-L328F, N325S-L328F, and H433K-N434F, relative to IgG1 Fc domain sequence SEQ ID NO: 6 or IgG4 Fc domain sequence SEQ ID NOS: 7 or 8 according to the EU numbering scheme. In embodiments, an exemplary SIRPα fusion polypeptide of the disclosure comprises an IgG4 human Fc domain of SEQ ID NO: 8 comprising substitutions M252Y, S254T, and T256E, relative to SEQ ID NO: 8. Exemplary SIRPα fusion polypeptides of the disclosure comprise the sequence of SEQ ID NO: 10, or an amino acid sequence with at least 70%, at least 75%, at least 80%, at least 85%, at least 90%, at least 95%, at least 96%, at least 97%, at least 98%, or at least 99% sequence identity thereto.

In some embodiments, inclusion of a modified Fc domain increases SIRPα fusion polypeptide induction of phagocytosis by a macrophage. In some embodiments, inclusion of the modified Fc domain increases SIRPα fusion polypeptide induction of phagocytosis by a macrophage by about about 1.5×, about 2×, about 3×, about 4×, about 5×, about 6×, about 7×, about 8×, about 9×, about 10×, about 20×, about 50×, about 100×, about 250×, about 500×, about 750×, about 1000×, about 5000× or about 10,000×, relative to that of a SIRPα fusion polypeptide comprising a wild type Fc domain. In some embodiments the one or more modification in the Fc domain is selected from the group consisting of T250Q-M428L, M252Y-S254T-T256E, M428L-N434S, S267E-L328F, N325S-L328F, and H433K-N434F, relative to IgG1 Fc domain sequence SEQ ID NO: 6 or IgG4 Fc domain sequence SEQ ID NOS: 7 or 8 according to the EU numbering scheme. In embodiments, an exemplary SIRPα fusion polypeptide of the disclosure comprises an IgG4 human Fc domain of SEQ ID NO: 8 comprising substitutions M252Y, S254T, and T256E, relative to SEQ ID NO: 8. Exemplary SIRPα fusion polypeptides of the disclosure comprise the sequence of SEQ ID NO: 10, or an amino acid sequence with at least 70%, at least 75%, at least 80%, at least 85%, at least 90%, at least 95%, at least 96%, at least 97%, at least 98%, or at least 99% sequence identity thereto.

In some embodiments, a SIRPα fusion polypeptide comprising a modified Fc domain is associated with increased degradation of a CD47 protein relative to a SIRPα fusion polypeptide comprising a wild type Fc domain. In some embodiments, inclusion of the modified Fc domain increases SIRPα fusion polypeptide associated degradation of a CD47 protein by about 1.5×, about 2×, about 3×, about 4×, about 5×, about 6×, about 7×, about 8×, about 9×, about 10×, about 20×, about 50×, about 100×, about 250×, about 500×, about 750×, about 1000×, about 5000× or about 10,000×, relative to that of a SIRPα fusion polypeptide comprising a wild type Fc domain. In some embodiments the one or more modification in the Fc domain is selected from the group consisting of T250Q-M428L, M252Y-S254T-T256E, M428L-N434S, S267E-L328F, N325S-L328F, and H433K-N434F, relative to IgG1 Fc domain sequence SEQ ID NO: 6 or IgG4 Fc domain sequence SEQ ID NOS: 7 or 8 according to the EU numbering scheme. In embodiments, an exemplary SIRPα fusion polypeptide of the disclosure comprises an IgG4 human Fc domain of SEQ ID NO: 8 comprising substitutions M252Y, S254T, and T256E, relative to SEQ ID NO: 8. Exemplary SIRPα fusion polypeptides of the disclosure comprise the sequence of SEQ ID NO: 10, or an amino acid sequence with at least 70%, at least 75%, at least 80%, at least 85%, at least 90%, at least 95%, at least 96%, at least 97%, at least 98%, or at least 99% sequence identity thereto.

In some embodiments, inclusion of a modified Fc domain decreases SIRPα fusion polypeptide toxicity. In some embodiments, inclusion of the modified Fc domain decreases SIRPα fusion polypeptide toxicity by about 1.5×, about 2×, about 3×, about 4×, about 5×, about 6×, about 7×, about 8×, about 9×, about 10×, about 20×, about 50×, about 100×, about 250×, about 500×, about 750×, about 1000×, about 5000× or about 10,000×, relative to that of a SIRPα fusion polypeptide comprising a wild type Fc domain. In some embodiments the one or more modification in the Fc domain is selected from the group consisting of T250Q-M428L, M252Y-S254T-T256E, M428L-N434S, S267E-L328F, N325S-L328F, and H433K-N434F, relative to IgG1 Fc domain sequence SEQ ID NO: 6 or IgG4 Fc domain sequence SEQ ID NOS: 7 or 8 according to the EU numbering scheme. In embodiments, an exemplary SIRPα fusion polypeptide of the disclosure comprises an IgG4 human Fc domain of SEQ ID NO: 8 comprising substitutions M252Y, S254T, and T256E, relative to SEQ ID NO: 8. Exemplary SIRPα fusion polypeptides of the disclosure comprise the sequence of SEQ ID NO: 10, or an amino acid sequence with at least 70%, at least 75%, at least 80%, at least 85%, at least 90%, at least 95%, at least 96%, at least 97%, at least 98%, or at least 99% sequence identity thereto.

In some embodiments, inclusion of a modified Fc domain decreases SIRPα fusion polypeptide antibody dependent cellular cytotoxicity (ADCC). In some embodiments, inclusion of the modified Fc domain decreases SIRPα fusion polypeptide antibody dependent cellular cytotoxicity (ADCC) by about 1.5×, about 2×, about 3×, about 4×, about 5×, about 6×, about 7×, about 8×, about 9×, about 10×, about 20×, about 50×, about 100×, about 250×, about 500×, about 750×, about 1000×, about 5000× or about 10,000×, relative to that of a SIRPα fusion polypeptide comprising a wild type Fc domain. In some embodiments the one or more modification in the Fc domain is selected from the group consisting of T250Q-M428L, M252Y-S254T-T256E, M428L-N434S, S267E-L328F, N325S-L328F, and H433K-N434F, relative to IgG1 Fc domain sequence SEQ ID NO: 6 or IgG4 Fc domain sequence SEQ ID NOS: 7 or 8 according to the EU numbering scheme. In embodiments, an exemplary SIRPα fusion polypeptide of the disclosure comprises an IgG4 human Fc domain of SEQ ID NO: 8 comprising substitutions M252Y, S254T, and T256E, relative to SEQ ID NO: 8. Exemplary SIRPα fusion polypeptides of the disclosure comprise the sequence of SEQ ID NO: 10, or an amino acid sequence with at least 70%, at least 75%, at least 80%, at least 85%, at least 90%, at least 95%, at least 96%, at least 97%, at least 98%, or at least 99% sequence identity thereto.

In some embodiments, inclusion of a modified Fc domain improves the stability of SIRPα fusion polypeptide associated Hemoglobin levels. In some embodiments, inclusion of the modified Fc domain improves the stability of SIRPα fusion polypeptide associated Hemoglobin levels by about about 1.5×, about 2×, about 3×, about 4×, about 5×, about 6×, about 7×, about 8×, about 9×, about 10×, about 20×, about 50×, about 100×, about 250×, about 500×, about 750×, about 1000×, about 5000× or about 10,000×, relative to that of a SIRPα fusion polypeptide comprising a wild type Fc domain. In some embodiments the one or more modification in the Fc domain is selected from the group consisting of T250Q-M428L, M252Y-S254T-T256E, M428L-N434S, S267E-L328F, N325S-L328F, and H433K-N434F, relative to IgG1 Fc domain sequence SEQ ID NO: 6 or IgG4 Fc domain sequence SEQ ID NOS: 7 or 8 according to the EU numbering scheme. In embodiments, an exemplary SIRPα fusion polypeptide of the disclosure comprises an IgG4 human Fc domain of SEQ ID NO: 8 comprising substitutions M252Y, S254T, and T256E, relative to SEQ ID NO: 8. Exemplary SIRPα fusion polypeptides of the disclosure comprise the sequence of SEQ ID NO: 10, or an amino acid sequence with at least 70%, at least 75%, at least 80%, at least 85%, at least 90%, at least 95%, at least 96%, at least 97%, at least 98%, or at least 99% sequence identity thereto.

In some embodiments, inclusion of a modified Fc domain decreases SIRPα fusion polypeptide associated anemia. In some embodiments, inclusion of the modified Fc domain decreases SIRPα fusion polypeptide associated anemia by about 1.5×, about 2×, about 3×, about 4×, about 5×, about 6×, about 7×, about 8×, about 9×, about 10×, about 20×, about 50×, about 100×, about 250×, about 500×, about 750×, about 1000×, about 5000× or about 10,000×, relative to that of a SIRPα fusion polypeptide comprising a wild type Fc domain. In some embodiments the one or more modification in the Fc domain is selected from the group consisting of T250Q-M428L, M252Y-S254T-T256E, M428L-N434S, S267E-L328F, N325S-L328F, and H433K-N434F, relative to IgG1 Fc domain sequence SEQ ID NO: 6 or IgG4 Fc domain sequence SEQ ID NOS: 7 or 8 according to the EU numbering scheme. In embodiments, an exemplary SIRPα fusion polypeptide of the disclosure comprises an IgG4 human Fc domain of SEQ ID NO: 8 comprising substitutions M252Y, S254T, and T256E, relative to SEQ ID NO: 8. Exemplary SIRPα fusion polypeptides of the disclosure comprise the sequence of SEQ ID NO: 10, or an amino acid sequence with at least 70%, at least 75%, at least 80%, at least 85%, at least 90%, at least 95%, at least 96%, at least 97%, at least 98%, or at least 99% sequence identity thereto.

In some embodiments, inclusion of a modified Fc domain decreases aggregation of SIRPα fusion polypeptides. In some embodiments, inclusion of the modified Fc domain decreases aggregation of SIRPα fusion polypeptides by about 1.5×, about 2×, about 3×, about 4×, about 5×, about 6×, about 7×, about 8×, about 9×, about 10×, about 20×, about 50×, about 100×, about 250×, about 500×, about 750×, about 1000×, about 5000× or about 10,000×, relative to that of a SIRPα fusion polypeptide comprising a wild type Fc domain. In some embodiments the one or more modification in the Fc domain is selected from the group consisting of T250Q-M428L, M252Y-S254T-T256E, M428L-N434S, S267E-L328F, N325S-L328F, and H433K-N434F, relative to IgG1 Fc domain sequence SEQ ID NO: 6 or IgG4 Fc domain sequence SEQ ID NOS: 7 or 8 according to the EU numbering scheme. In embodiments, an exemplary SIRPα fusion polypeptide of the disclosure comprises an IgG4 human Fc domain of SEQ ID NO: 8 comprising substitutions M252Y, S254T, and T256E, relative to SEQ ID NO: 8. Exemplary SIRPα fusion polypeptides of the disclosure comprise the sequence of SEQ ID NO: 10, or an amino acid sequence with at least 70%, at least 75%, at least 80%, at least 85%, at least 90%, at least 95%, at least 96%, at least 97%, at least 98%, or at least 99% sequence identity thereto.

In some embodiments, inclusion of a modified Fc domain improves the purification of SIRPα fusion polypeptides. In some embodiments, inclusion of the modified Fc domain improves the purification of SIRPα fusion polypeptides by about 1.5×, about 2×, about 3×, about 4×, about 5×, about 6×, about 7×, about 8×, about 9×, about 10×, about 20×, about 50×, about 100×, about 250×, about 500×, about 750×, about 1000×, about 5000× or about 10,000×, relative to that of a SIRPα fusion polypeptide comprising a wild type Fc domain. In some embodiments the one or more modification in the Fc domain is selected from the group consisting of T250Q-M428L, M252Y-S254T-T256E, M428L-N434S, S267E-L328F, N325S-L328F, and H433K-N434F, relative to IgG1 Fc domain sequence SEQ ID NO: 6 or IgG4 Fc domain sequence SEQ ID NOS: 7 or 8 according to the EU numbering scheme. In embodiments, an exemplary SIRPα fusion polypeptide of the disclosure comprises an IgG4 human Fc domain of SEQ ID NO: 8 comprising substitutions M252Y, S254T, and T256E, relative to SEQ ID NO: 8. Exemplary SIRPα fusion polypeptides of the disclosure comprise the sequence of SEQ ID NO: 10, or an amino acid sequence with at least 70%, at least 75%, at least 80%, at least 85%, at least 90%, at least 95%, at least 96%, at least 97%, at least 98%, or at least 99% sequence identity thereto.

In some embodiments, inclusion of a modified Fc domain reduces the formation of antidrug antibodies (ADA) by SIRPα fusion polypeptides. In some embodiments, inclusion of the modified Fc domain reduces the formation of ADA by SIRPα fusion polypeptides by about 1 about 1.5×, about 2×, about 3×, about 4×, about 5×, about 6×, about 7×, about 8×, about 9×, about 10×, about 20×, about 50×, about 100×, about 250×, about 500×, about 750×, about 1000×, about 5000× or about 10,000×, relative to that of a SIRPα fusion polypeptide comprising a wild type Fc domain. In some embodiments the one or more modification in the Fc domain is selected from the group consisting of T250Q-M428L, M252Y-S254T-T256E, M428L-N434S, S267E-L328F, N325S-L328F, and H433K-N434F, relative to IgG1 Fc domain sequence SEQ ID NO: 6 or IgG4 Fc domain sequence SEQ ID NOS: 7 or 8 according to the EU numbering scheme. In embodiments, an exemplary SIRPα fusion polypeptide of the disclosure comprises an IgG4 human Fc domain of SEQ ID NO: 8 comprising substitutions M252Y, S254T, and T256E, relative to SEQ ID NO: 8. Exemplary SIRPα fusion polypeptides of the disclosure comprise the sequence of SEQ ID NO: 10, or an amino acid sequence with at least 70%, at least 75%, at least 80%, at least 85%, at least 90%, at least 95%, at least 96%, at least 97%, at least 98%, or at least 99% sequence identity thereto.

In some embodiments, inclusion of a modified Fc domain reduces SIRPα fusion polypeptide associated red blood cell (RBC) agglutination. In some embodiments, inclusion of the modified Fc domain reduces SIRPα fusion polypeptide associated RBC agglutination by about 1.5×, about 2×, about 3×, about 4×, about 5×, about 6×, about 7×, about 8×, about 9×, about 10×, about 20×, about 50×, about 100×, about 250×, about 500×, about 750×, about 1000×, about 5000× or about 10,000×, relative to that of a SIRPα fusion polypeptide comprising a wild type Fc domain. In some embodiments the one or more modification in the Fc domain is selected from the group consisting of T250Q-M428L, M252Y-S254T-T256E, M428L-N434S, S267E-L328F, N325S-L328F, and H433K-N434F, relative to IgG1 Fc domain sequence SEQ ID NO: 6 or IgG4 Fc domain sequence SEQ ID NOS: 7 or 8 according to the EU numbering scheme. In embodiments, an exemplary SIRPα fusion polypeptide of the disclosure comprises an IgG4 human Fc domain of SEQ ID NO: 8 comprising substitutions M252Y, S254T, and T256E, relative to SEQ ID NO: 8. Exemplary SIRPα fusion polypeptides of the disclosure comprise the sequence of SEQ ID NO: 10, or an amino acid sequence with at least 70%, at least 75%, at least 80%, at least 85%, at least 90%, at least 95%, at least 96%, at least 97%, at least 98%, or at least 99% sequence identity thereto.

In some embodiments, inclusion of a modified Fc domain reduces the formation of antibodies to red blood cells associated with a SIRPα fusion polypeptide as measured by the Coombs assay. In some embodiments, inclusion of the modified Fc domain reduces the formation of antibodies to red blood cells associated with a SIRPα fusion polypeptide by about 1.5×, about 2×, about 3×, about 4×, about 5×, about 6×, about 7×, about 8×, about 9×, about 10×, about 20×, about 50×, about 100×, about 250×, about 500×, about 750×, about 1000×, about 5000× or about 10,000×, relative to that of a SIRPα fusion polypeptide comprising a wild type Fc domain. In some embodiments the one or more modification in the Fc domain is selected from the group consisting of T250Q-M428L, M252Y-S254T-T256E, M428L-N434S, S267E-L328F, N325S-L328F, and H433K-N434F, relative to IgG1 Fc domain sequence SEQ ID NO: 6 or IgG4 Fc domain sequence SEQ ID NOS: 7 or 8 according to the EU numbering scheme. In embodiments, an exemplary SIRPα fusion polypeptide of the disclosure comprises an IgG4 human Fc domain of SEQ ID NO: 8 comprising substitutions M252Y, S254T, and T256E, relative to SEQ ID NO: 8. Exemplary SIRPα fusion polypeptides of the disclosure comprise the sequence of SEQ ID NO: 10, or an amino acid sequence with at least 70%, at least 75%, at least 80%, at least 85%, at least 90%, at least 95%, at least 96%, at least 97%, at least 98%, or at least 99% sequence identity thereto.

### III. Methods of Making SIRPα Fusion Polypeptides Comprising a Modified Fc Domain

Also provided herein are polynucleotides encoding the SIRPα fusion polypeptides comprising modified Fc domains for improved pharmacokinetics of the disclosure. In some embodiments, the polynucleotide encodes any of the aforementioned SIRPα fusion polypeptides, or a sequence with at least 70%, at least 80%, at least 90%, at least 95%, at least 98%, or at least 99% sequence identity thereto.

In some embodiments, a polynucleotide encoding an exemplary SIRPα fusion polypeptide of the disclosure is introduced (e.g., transfected or transformed) and expressed in a human cell line, or a bacterial cell line. Exemplary cell lines available for production include, but are not limited to, Expi 293 and CHO cell lines.

### IV. Methods of Using SIRPα Fusion Polypeptides Comprising a Modified Fc Domain

In some embodiments, a SIRPα fusion polypeptide comprising a modified Fc domain is administered to a subject in need thereof as a therapeutic. In some embodiments, a SIRPα fusion polypeptide of the disclosure is administered to treat, for example, a cardiovascular disease, a cancer, fibrosis, an infectious disease, a hematological disease, or a neurological disease. In some embodiments, inclusion of a modified Fc domain improves the efficacy and/or safety of a SIRPα fusion polypeptide as a therapeutic.

In some embodiments, a subject selected for treatment with a SIRPα fusion polypeptide has a cardiovascular disease, and has or is determined to be at risk of having, one or more of and has or is determined to be at risk of having, one or more of atherosclerosis, heart failure, myocardial infarction, cardiomyopathy, acute coronary syndrome, myocarditis, cardiac remodeling, hypertension, angina, restenosis, stroke, aneurysms, thrombosis, phlebitis, peripheral vascular disease, pulmonary arterial hypertension, and autoimmune vasculitis.

In some embodiments, a subject selected for treatment with a SIRPα fusion polypeptide has a cancer. In some embodiments, a SIRPα fusion polypeptide of the disclosure may treat tumor growth and/or tumor metastasis, e.g., of a lymphoma, leukemia, carcinoma, melanoma, glioblastoma, sarcoma, or myeloma.

In some embodiments, a subject selected for treatment with a SIRPα fusion polypeptide of the disclosure has fibrosis or a fibrotic disease, e.g., a liver or lung fibrotic disease. In some embodiments, the subject has or is at risk of having end-stage liver disease, kidney disease, idiopathic pulmonary fibrosis (IPF), retinal fibrosis, chronic graft rejection from progressive myopathy, or heart failure from cardiac fibrosis.

In some embodiments, a subject selected for treatment with a SIRPα fusion polypeptide has an infectious disease has an infectious disease associated with a virus, bacteria, or fungal pathogen. In some embodiments the subject has a viral infections, e.g., an infection associated with one of a retrovirus, lentivirus, hepadna virus, herpes virus, pox virus, or human papilloma virus. In some embodiments, the subject has an intracellular bacterial infections, e.g., an infection associated with one of Mycobacterium, Chlamydophila, Ehrlichia, Rickettsia, Brucella, Legionella, Francisella, Listeria, Coxiella, Neisseria, Salmonella, or Yersinia species. In some embodiments, the subject has an intracellular protozoan pathogen infection, e.g., an infection associated with one of a Plasmodium species, Trypanosoma species, Giardia species, Toxoplasma species, or Leishmania species.

In some embodiments, a subject is selected for treatment with a SIRPα fusion polypeptide of the disclosure with a hematological disease or disorder, e.g. a genetic blood disorder or severe combined immunodeficiency. In some embodiments, a SIRPα fusion polypeptide of the disclosure may be used alone or in combination with other agents to facilitate engraftment of endogenous stem cells prior to hematopoietic stem cell transplant.

In some embodiments, a subject selected for treatment with a SIRPα fusion polypeptide has a neurological disease.

In some embodiments, a SIRPα fusion polypeptide may be delivered to a subject in need thereof subcutaneously, intravenously, intravitreally, orally, intranasally, transdermaly, intraperitoneally, intramuscularly, intrathecally, intrapulmonary, vaginally, or rectally. In some embodiments, the SIRPα fusion polypeptide is administered subcutaneously.

In some embodiments, a SIRPα fusion polypeptide is conjugated to a fluorophore, a radionucleotide or other imaging or diagnostic moiety. In some embodiments, a SIRPα fusion polypeptide is administered to a cell or an organism to image the position or concentration of a CD47 protein. In some embodiments, a SIRPα fusion polypeptide is administered to a cell or an organism to diagnose a disease.

In some embodiments, the SIRPα fusion polypeptide comprises a conjugated toxin to deliver the toxin to a cell expressing CD47.

In some embodiments, a SIRPα fusion polypeptide is administered in combination with a CD20 antibody and/or a CD47 antibody, or fragment thereof. In some embodiments, a multivalent SIRPα fusion polypeptide is administered in combination with an antibody, or antibody fragment, to a protein selected from the group comprising: TNF alpha, TNF-alpha R, IL6, IL6R, IL1 beta, IL1-beta R, IL17A, CD117, EGFR, HER2, CD20, PD1/PDL1, CD137, CTLA4, LAG3, CD3, CD2, CD4, CD19, CD38, GD2, VEGF, VEGF-R, P-selectin, CCR4, CD52, IL2, and IL2 R.

### VI. Pharmaceutical Compositions

In some embodiments, a SIRPα fusion polypeptide comprising a modified Fc domain is present in a pharmaceutical composition. In particular embodiments, the pharmaceutical compositions may be in a water-soluble form, such as in pharmaceutically acceptable salts, which is meant to include both acid and base addition salts. Pharmaceutically acceptable acid addition salts include but are not limited to: hydrochloric acid, hydrobromic acid, sulfuric acid, nitric acid, phosphoric acid, organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, and salicylic acid. Pharmaceutically acceptable base addition salts include those derived from inorganic bases such as sodium, potassium, lithium, ammonium, calcium, magnesium, iron, zinc, copper, manganese, aluminum salts and the like. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

Pharmaceutical compositions as described herein may also include one or more of the following: carrier proteins such as serum albumin; buffers; fillers such as microcrystalline cellulose, lactose, corn and other starches; binding agents; and polyethylene glycol.

The compositions for administration will commonly include the polypeptide dissolved in a pharmaceutically acceptable carrier, preferably an aqueous carrier. A variety of aqueous carriers can be used, e.g., buffered saline. The composition may contain pharmaceutically acceptable auxiliary substances as required to approximate physiological conditions such as pH and buffering agents, toxicity countering agents, e.g., sodium acetate, sodium chloride, potassium chloride, calcium chloride, and sodium lactate. The concentration of active agents in the formulations can vary and are selected based on fluid volumes, viscosities, and body weight in accordance with the particular mode of administration selected and the patient's needs (e.g., Remington's Pharmaceutical Science (15th ed., 1980) and Goodman & Gillman, The Pharmacological Basis of Therapeutics (Hardman et al., eds., 1996)).

### VII. Kits

A SIRPα fusion polypeptide comprising a modified Fc domain as described herein may also comprise a therapeutic or diagnostic kit for administration by a medical professional or the subject in need thereof. The kit may comprise for example, a container, a dose of a SIRPα fusion polypeptide, a syringe and/or a vial, and instructions for use thereof. In some embodiments the kit comprises a SIRPα fusion polypeptide and instructions for administering the polypeptide to treat a disease.

In some embodiments, the SIRPα fusion polypeptide of a kit is conjugated to a fluorophore, a radionucleotide or other diagnostic moiety.

### EXAMPLES

### Example 1: The Effect of the YTE Mutation in a SIRPα Fusion Polypeptide on CD47 Finding Affinity

To determine the effects of the YTE Fc domain mutation on CD47 binding affinity, CD47 binding experiments were performed for a SIRPα fusion polypeptide of SEQ ID NO: 10, which comprises the SIRPα domain of SEQ ID NO: 2 and an IgG4 Fc domain with a YTE mutation (Construct 67). Also measured were the CD47 binding affinity of the SIRPα fusion polypeptide SEQ ID NO: 10 construct without the YTE mutation (Construct 50), i.e, with the wild type IgG4 Fc domain; and the binding affinity of MIAP410, which is a commercially available CD47 monoclonal antibody. MIAP410 is a mouse anti-human CD47 antibody that reacts with human, mouse, and rat CD47.

A Sartorius Biosensor Octet® R8 was used to measure an affinity octet of the aforementioned constructs and antibody with AMC (anti-mouse Fc-capture) Catalogue No. 18-5088 or AHC (anti-human Fc-capture) Catalogue No. 18-5060.

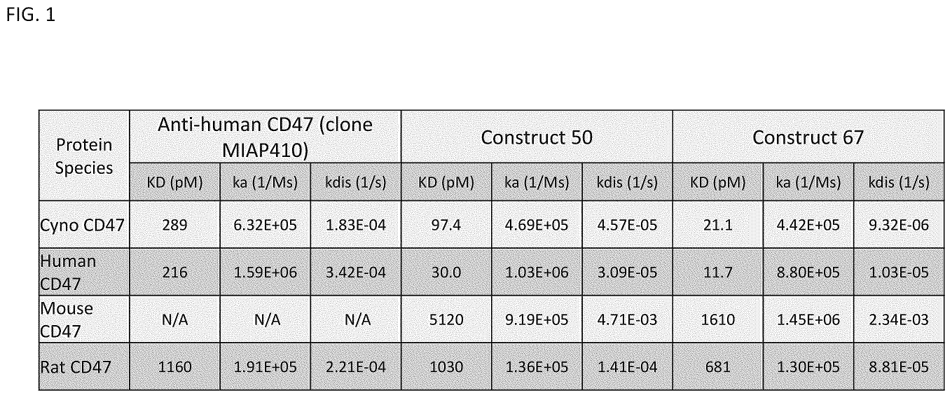

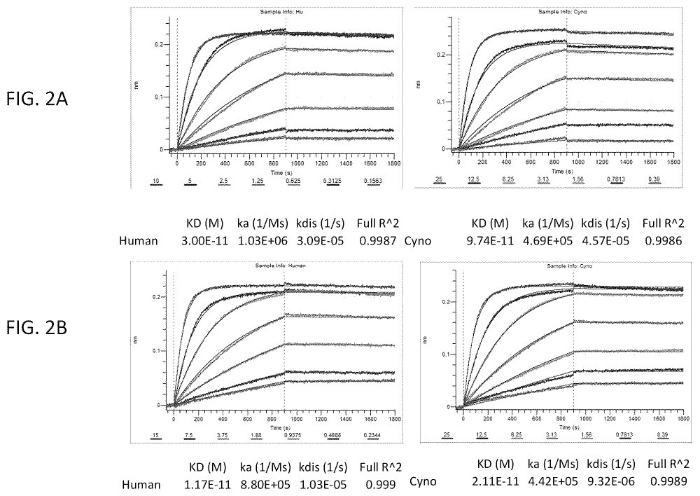

The binding affinity assays were performed at 30° C. using the following method. The running buffer was PBS, 1% BSA, and 0.05% Tween-20. The SIRPα fusion polypeptides and CD47 antibody were immobilized to AMC or AHC for 3 minutes, followed by rhCD47-his association and dissociation for 15 minutes. Data was analyzed with the Sartorius software Octet Analysis Studio using the values after reference subtraction. The disassociation constant (KD) was determined by applying a global (full kinetics) 1:1 fitting model (N=1 per sample for the SIRPα fusion polypeptides and N=5 for the anti-CD47 antibody); Data from the best kinetic curve fit experiment is presented in FIG. 1, and the example of individual run results are shown in FIG. 2A (Construct 50) and FIG. 2B (Construct 67).

The data demonstrate that both Construct 67, which is the SIRPα fusion polypeptide of SEQ ID NO: 10, and Construct 50 which is the same construct as Construct 67, but without the YTE mutation, have significantly higher binding affinities to all CD47 proteins tested (Cyno monkey, human, mouse, and rat), relative to the binding affinity of the the MIAP410 anti-CD47 antibody. Notably, Construct 67, which contains the YTE mutation in the IgG4 Fc domain, demonstrated a binding affinity of equal or better than that of the Construct 50 SIRPα fusion polypeptide, which does not contain the YTE mutation.

### Example 2: The Effect of the YTE Mutation in a SIRPα Fusion Polypeptide on In Vitro Human Red Blood Cell Binding

To determine the effects of the YTE mutation on red blood cell binding of the SIRPα fusion polypeptide of SEQ ID NO: 10 (Construct 67), the following experiments were performed with Construct 67 and with Construct 50 as a comparison.

Normal human whole blood in EDTA from two different donors were purchased from Stanford Blood Center and shipped overnight at room temperature. Cells were incubated with 0.18 to 400 nM of Construct 50 or Construct 67 for 20 minutes at 4° C. Cells were washed with PBS for three times followed by incubation with AF488 conjugated anti-human CD45, and 30 μg/mL AF647-labeled anti-human IgG4 antibody for 20 minutes at 4° C. Post washing, binding was measured using an Agilent Quanteon using CD45 gating (N=2 donors) to select for the effect in red blood cells.

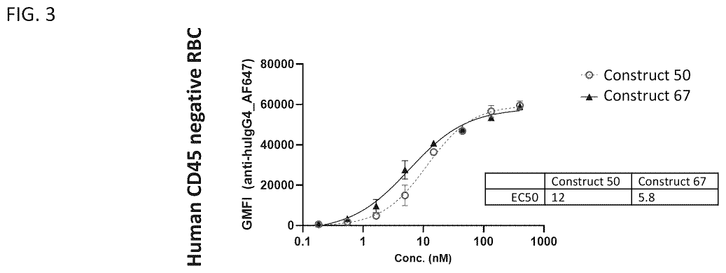

The EC₅₀ value measured for the two constructs was the amount of construct bound to red blood cells in vitro. As shown in FIG. 3, at half-maximal binding of Construct 67, which contains the YTE mutation, was equal or lower than that of Construct 50.

### Example 3: The Effect of the YTE Mutation in a SIRPα Fusion Polypeptide on In Vitro Phagocytosis

To determine the effects of the YTE mutation in a SIRPα fusion polypeptide on the induction of in vitro phagocytosis, the following experiments were performed using Construct 67 containing the YTE Fc domain mutation, Construct 50 containing the wild type Fc domain, and biosimilar anti-CD47 magrolimab-like antibody.

Eight days prior to running the assay, healthy human buffy coat from the LRS chamber purchased from Stanford Blood Center and shipped overnight. PBMC cells were isolated from buffy coat by applying ficoll-paque gradient, and monocytes were isolated using EasySep human monocyte isolation kit purchased from Stemcell (Cat #19359). Isolated monocytes were plated and differentiated for 7 days in IMDM, 10% human serum (from Innovative Research), and 1% penicillin and streptomycin. On the day of experiment, Raji cells were first labeled with calcein using the following protocol. Calcein-AM dye were reconstituted in 20 μl DMSO and added to Raji cells (1 μl calcein-DMSO solution for every 5×10⁶ cells) and incubated at 37° C. for 10 mins followed by incubation on ice for 10 mins. Raji cells were then centrifuged at 1250 rpm for 5 mins at 4° C. The cells were washed once with HBSS and resuspended in IMDM. 25 μl/well of the calcein stained Raji (target) cells suspended in IMDM at 4×10⁶ cells/ml density were pipetted into a Costar ultra-low attachment 96 well plate. 25 μl/well of biosimilar Magrolimab-like antibody, Construct 50 and Construct 67 at concentrations of 0.67 nM and 6.67 nM and 6.67 nM IgG4 isotype control in IMDM was added to Raji cells in triplicates. The Raji cells were pre-incubated with the test articles at 37° C. for 30 mins. 50 μl/well of in vitro differentiated macrophages from human donors in IMDM at 1×10⁶ cells/ml were added in triplicates to the target cells. The cells were resuspended and incubated at 37° C. for 2 hrs. At the end of incubation, the plate was centrifuged, and the supernatant was discarded. The cells were then resuspended in 50 μl of PBS+2% FBS+5 ug/ml Alexa Fluor® 647 anti-human CD206 and incubated on ice and in dark for 30 mins. The cells were washed once with 200 μl FACS buffer and resuspended in 100 μl of FACS buffer+DAPI. The phagocytosis index was detected using Flow cytometer and analyzed by Flowjo software. Percent macrophages is the percent macrophages having phagocytosed calcein-AM-labeled Raji.

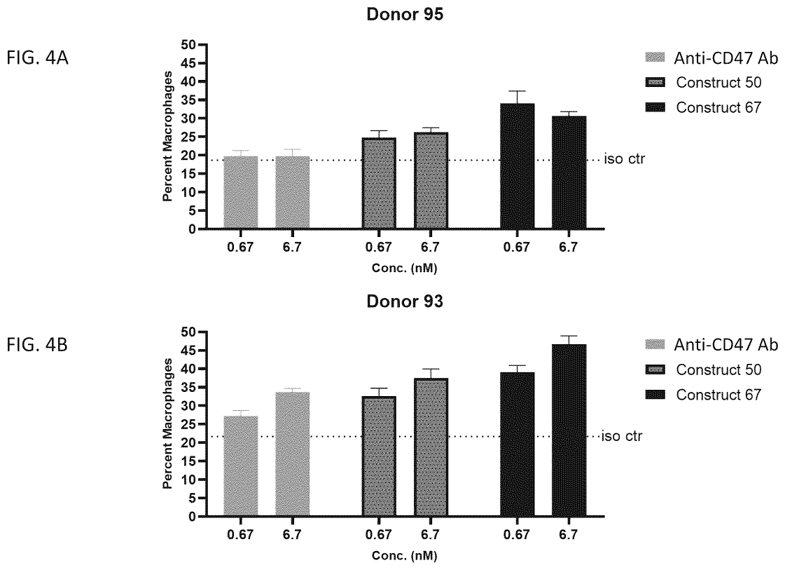

As shown in FIGS. 4A and 4B, Construct 50 and Construct 67 induced an increased level of phagocytoxis relative to biosimilar Magrolimab-like antibody. Further, Construct 67 which contains the YTE Fc domain, induced an equal or better level of phagocytosis than did Construct 50, which lacks the YTE mutation in the IgG4 Fc domain.

### Example 4: The Effect of the YTE Mutation in a SIRPα Fusion Polypeptide on In Vitro Antibody Dependent Cellular Cytotoxicity (ADCC)

To determine the effects of the YTE mutation in the SIRPα fusion polypeptides on the induction of antibody dependent cellular cytotoxicity (ADCC), the following experiments were performed using Construct 67 with the YTE Fc domain mutation and Construct 50 with the wild type Fc domain, and an anti-CD20 antibody, as a positive control.

ADCC assay was performed according to the instructions from ADCC Reporter Bioassay Complete Kit purchased from Promega (Cat #G7015). In brief, 25 μl/well of Raji cells in RPMI 1640 in low IgG serum media, prepared according to the kit instructions, were pipetted into a white flat bottomed 96 well Corning 3610 microplate. Serial dilutions of anti-CD20 (14 pM to 10 nM), Construct 50 and Construct 67 (457 pM to 1 μM), and 1 μM IgG4 were prepared in RPMI 1640 in low IgG serum. 25 μl/well of antibody/molecule were pipetted into the wells containing target cells with one set of wells receiving no antibody/molecule. IgG4 and anti-CD20 was used as negative and positive controls respectively. To the wells containing the target Raji cells and drug/molecule, 25 μl/well of effector cells prepared in RPMI 1640 in low IgG serum was pipetted. The microplate incubated at 37° C. for 6 hours, followed by luminescence detection using reagents supplied with the kit.

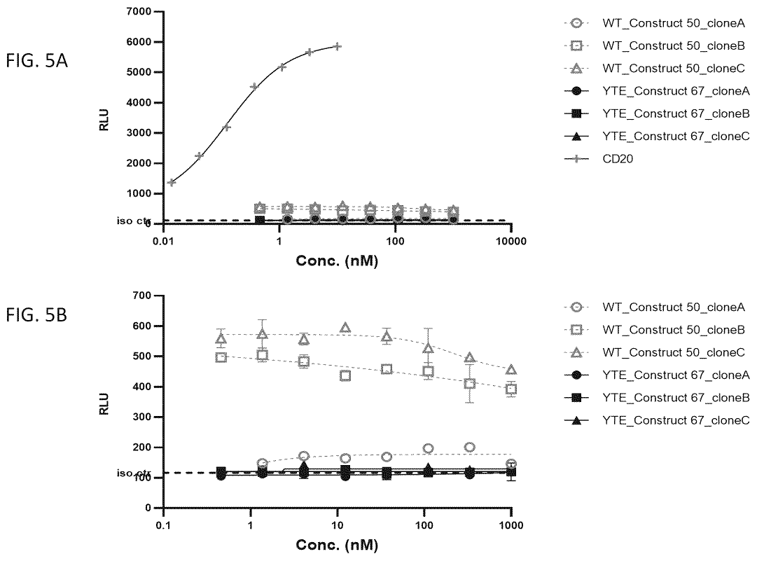

As shown in FIG. 5A, Construct 67 and Construct 50 demonstrated minimal ADCC compared to the anti-CD20 antibody. The inset of FIG. 5A, is shown in FIG. 5B, which is rescaled to show the difference between Construct 67 containing the YTE mutation in the Fc domain and Construct 50, containing the wild type Fc domain. The data demonstrate that the YTE containing Construct 67 exhibited less antibody-dependent cellular cytotoxicity than did Construct 50.

### Example 5: The Effect of the YTE Mutation on In Vivo Receptor Occupancy, Hemoglobin Levels, and Pharmacokinetics of a SIRPα Fusion Polypeptide in Non-Human Primates

To determine the effects of the YTE mutation in SIRPα fusion polypeptides in vivo receptor occupancy, hemoglobin levels, and pharmacokinetics (PK), the following experiments are performed using a construct of the disclosure (e.g. Construct 67 with a YTE Fc domain substitution) and wild type construct (e.g. Construct 50 with a wild type Fc domain). Biological naive male cynomolgus non-human primates are dosed by subcutaneous dosing and/or intravenous dosing. Receptor occupancy, hemoglobin, and/or PK data are collected and analyzed before, during, and after administration, and over a period of observation. Analysis may be gated for CD45 negative cells.

It is predicted that the receptor occupancy of red blood cells treated with certain constructs of the disclosure, comprising the YTE Fc domain substitution, is higher or the same as the receptor occupancy of red blood cells treated with a wild type construct over the course of the observation period.

It is predicted that the blood serum concentration of a subject with certain constructs of the disclosure, comprising the YTE Fc domain substitution, is higher or the same as the blood serum concentration of a subject treated with a wild type construct over the course of the observation period.

It is predicted that hemoglobin levels of a subject with certain constructs of the disclosure, comprising the YTE Fc domain substitution, is higher or the same as hemoglobin levels of a subject treated with a wild type construct over the course of the observation period.

1. A fusion polypeptide comprising a signal-regulatory protein α (SIRPα) domain, and a modified Fc domain, wherein the modified Fc domain comprises one or more amino acid modifications relative to a wild type Fc domain, and wherein the inclusion of the modified Fc domain increases binding affinity to FcRn.

2. The fusion polypeptide of claim 1, wherein the inclusion of the modified Fc domain increases the half-life of the fusion polypeptide.

3. The fusion polypeptide of claim 1, wherein the inclusion of the modified Fc domain increases the blood clearance time of the fusion polypeptide.

4. The fusion polypeptide of claim 1, wherein the inclusion of the modified Fc domain improves the binding affinity of the fusion polypeptide to a CD47 protein.

5. The fusion polypeptide of claim 1, wherein the inclusion of the modified Fc domain lowers the Ec50 of the fusion polypeptide.

6. The fusion polypeptide of claim 1, wherein the inclusion of the modified Fc domain lowers the effective dose of the fusion polypeptide necessary to achieve a therapeutic effect in a subject.

7. The fusion polypeptide of claim 1, wherein the inclusion of the modified Fc domain lowers the dosage frequency of the fusion polypeptide in a subject.

8. The fusion polypeptide of claim 1, wherein the inclusion of the modified Fc domain decreases the toxicity of the fusion polypeptide.

9. The fusion polypeptide of claim 8, wherein the inclusion of the modified Fc domain decreases the antibody dependent cellular cytotoxicity (ADCC).

10. The fusion polypeptide of claim 1, wherein the inclusion of the modified Fc domain increases effector function of the fusion polypeptide.

11. The fusion polypeptide of claim 1, wherein the inclusion of the modified Fc domain increases phagocytosis by a macrophage.

12. The fusion polypeptide of claim 1, wherein the inclusion of the modified Fc domain increases the interaction of the fusion polypeptide with a cell expressing a CD47 protein.

13. The fusion polypeptide of claim 1, wherein the inclusion of the modified Fc domain increases endocytosis of a CD47 protein.

14. The fusion polypeptide of claim 1, wherein the inclusion of the modified Fc domain increases degradation of a CD47 protein.

15. The fusion polypeptide of claim 12, wherein the CD47 protein is a human or mouse CD47 protein.

16. The fusion polypeptide of claim 1, wherein the inclusion of the modified Fc domain reduces aggregation of the fusion polypeptide.

17. The fusion polypeptide of claim 1, wherein the inclusion of the modified Fc domain improves purification of the polypeptide.

18. The fusion polypeptide of claim 1, wherein the modified Fc domain comprises the IgG4 Fc domain amino acid sequence of SEQ ID NOS: 7 or 8 or an amino acid sequence with at least 70%, at least 75%, at least 80%, at least 85%, at least 90%, at least 95%, at least 96%, at least 97%, at least 98%, or at least 99% sequence identity thereto.

19. The fusion polypeptide of claim 18, wherein the modified Fc domain comprises one or more substitutions selected from the group consisting of T250Q, M252Y, S254T, T256E, S267E, N325S, L328F, N343S, M428L, N434F, and H443K relative to SEQ ID NOS: 7 or 8, according to the EU numbering scheme.

20. The fusion polypeptide of claim 18, wherein the modified Fc domain comprises one or more substitutions selected from the group consisting of T250Q-M428L, M252Y-S254T-T256E, M428L-N434S, S267E-L328F, N325S-L328F, and H433K-N434F, relative to SEQ ID NOS: 7 or 8 according to the EU numbering scheme.

21. The fusion polypeptide of claim 1, wherein the modified Fc domain comprises SEQ ID NO: 9 or an amino acid sequence with at least 70%, at least 75%, at least 80%, at least 85%, at least 90%, at least 95%, at least 96%, at least 97%, at least 98%, or at least 99% sequence identity thereto.

22. The fusion polypeptide of claim 1, wherein the modified Fc domain comprises the IgG1 Fc domain amino acid sequence of SEQ ID NO: 6 or an amino acid sequence with at least 70%, at least 75%, at least 80%, at least 85%, at least 90%, at least 95%, at least 96%, at least 97%, at least 98%, or at least 99% sequence identity thereto.

23. The fusion polypeptide of claim 22, wherein the modified Fc domain comprises one or more substitutions selected from the group consisting of V215A, G236A, S239D, and I332E relative to SEQ ID NO: 6, according to the EU numbering scheme.

24. The fusion polypeptide of claim 22, wherein the modified Fc domain comprises one or more substitutions selected from the group consisting of T250Q, M252Y, S254T, T256E, S267E, N325S, L328F, N343S, M428L, H433K, and N434F relative to SEQ ID NO: 6, according to the EU numbering scheme.

25. The fusion polypeptide of claim 22, wherein the modified Fc domain comprises one or more substitutions selected from the group consisting of T250Q-M428L, M252Y-S254T-T256E, M428L-N434S, S267E-L328F, N325S-L328F, and H433K-N434F relative to SEQ ID NO: 6, according to the EU numbering scheme.

26. The fusion polypeptide of claim 1, wherein the modified Fc domain is a modified human IgG1, IgG2, IgG3, or IgG4 domain.

27. The fusion polypeptide of claim 1, wherein the SIRPα domain comprises the amino acid sequence of SEQ ID NO: 1 or an amino acid sequence with at least 70%, at least 75%, at least 80%, at least 85%, at least 90%, at least 95%, at least 96%, at least 97%, at least 98%, or at least 99% sequence identity thereto.

28. The fusion polypeptide of claim 1, wherein the SIRPα domain comprises one or more of the following mutations relative to SEQ ID NO: 1: V6I, S14L, S20T, I22T, H24R, V27I, I31F, A45G, E47V, K53R, E54Q, H56P, S66T, E70N, S77R, V92I, and/or a duplication of the D100 residue.

29. The fusion polypeptide of claim 1, wherein the SIRPα domain comprises the amino acid sequence of SEQ ID NO: 2 or an amino acid sequence with at least 70%, at least 75%, at least 80%, at least 85%, at least 90%, at least 95%, at least 96%, at least 97%, at least 98%, or at least 99% sequence identity thereto.

30. The fusion polypeptide of claim 1, wherein the modified Fc domain comprises any of SEQ ID NOS: 6-9, or an amino acid sequence with at least 70%, at least 75%, at least 80%, at least 85%, at least 90%, at least 95%, at least 96%, at least 97%, at least 98%, or at least 99% sequence identity thereto; and the SIRPα domain comprises SEQ ID NO: 1 or SEQ ID NO: 2 or an amino acid sequence with at least 70%, at least 75%, at least 80%, at least 85%, at least 90%, at least 95%, at least 96%, at least 97%, at least 98%, or at least 99% sequence identity thereto.

31. The fusion polypeptide of claim 1, wherein the modified Fc domain comprises SEQ ID NO: 9 or an amino acid sequence with at least 70%, at least 75%, at least 80%, at least 85%, at least 90%, at least 95%, at least 96%, at least 97%, at least 98%, or at least 99% sequence identity thereto; and the SIRPα domain comprises SEQ ID NO: 2 or an amino acid sequence with at least 70%, at least 75%, at least 80%, at least 85%, at least 90%, at least 95%, at least 96%, at least 97%, at least 98%, or at least 99% sequence identity thereto.

32. A method of treating a disease in a subject in need thereof, comprising administering a fusion polypeptide of claim 1 to the subject.

33. The method of claim 32, wherein the disease is a cardiovascular disease.

34. The method of claim 32, wherein the fusion polypeptide is administered subcutaneously.

35. A method of increasing phagocytosis by macrophages comprising contacting a population of macrophages with the fusion polypeptide of claim 1.

36. The method of claim 35, wherein the macrophage is a human macrophage.

37. A nucleotide encoding the fusion polypeptide of claim 1.
